(19) United States
(12) Patent Application Publication
(10) Pub. No.: US 2008/0200673 A1
(43) Pub. Date: Aug. 21, 2008

(54) Process for The Preparation of Sulfonic Acid Salts of Oxabispidines

(75) Inventors: Lal Cheema, Leicestershire (GB); David Cladingboel, Leicestershire (GB)

Correspondence Address: Pepper Hamilton LLP, 400 Berwyn Park, 899 Cassatt Road, Berwyn, PA, 19312-1183, US

(73) Assignee: AstraZeneca AB, Sodertalje (SE)

(21) Appl. No.: 11/993,026

(22) PCT Filed: Jun. 12, 2006
(86) PCT No.: PCT/SE06/00691

§ 371 Date: Feb. 8, 2008

(30) Foreign Application Priority Data
Jun. 20, 2005 (SE) 0501426-1
Dec. 15, 2005 (SE) 0502773-5

(51) Int. Cl. C07D 498/08

(52) U.S. Cl. 544/74

(57) ABSTRACT

There is provided a process for the isolation of a sulfonic acid salt of formula I, or a solvate thereof, from a mixture comprising: (i) the corresponding free base; and (ii) a compound of formula III. or a salt and/or solvate thereof, which process comprises providing an aqueous dispersion of the compounds of formulae II and III and a source of R³SO₃_{^{−}} anions and then, if necessary, adjusting the pH of the aqueous dispersion to any value from 3 to 8. There are further provided processes wherein the mixture of compounds of formulae II and III is provided by incomplete reaction, for example in the presence of base and an aqueous phase, between a compound of formula III and a compound of formula IV In such processes, the RSO₃_{^{−}} anions of the resulting salt of formula I may be derived from the compound of formula IV. Also for all of these processes, D, R¹, R² and R³ have meanings given in the description.

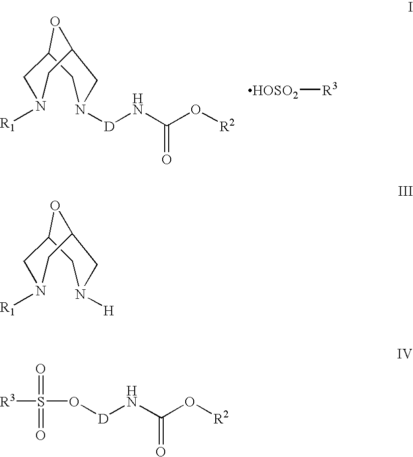

### FIELD OF THE INVENTION

The invention relates to a novel process for the preparation of sulfonic acid salts of oxabispidines that bear a N-(alkoxycarbonylamino)alkyl substituent.

### BACKGROUND AND PRIOR ART

In the preparation of drug substances, it is desirable for the level of impurities (i.e. materials other than the desired active substance) to be kept to the minimum possible level.

Impurities that can be particularly problematic include by-products from the synthesis of the active substance, as these by-products can be closely related (in structural terms) to that substance. Structural similarity between the active substance and the by-product may mean that:
(a) the active substance and the by-product have very similar physical and chemical properties, and are hence very difficult to separate; and/or
(b) the by-product has pharmacological activity that is unwanted and potentially harmful.

International patent application WO 01/028992 describes the synthesis of a wide range of oxabispidine compounds, which compounds are indicated as being useful in the treatment of cardiac arrhythmias. Amongst the compounds disclosed are a number that bear a N-2-(tert-butoxycarbonylamino)ethyl substituent. International patent applications WO 02/028864 and WO 02/083690 disclose new processes for the synthesis of oxabispidine-based compounds, including certain compounds that bear a N-2-(alkoxycarbonylamino)ethyl substituent.

However, the above-mentioned documents do not disclose any methods that allow for the selective precipitation of a sulfonate salt of an oxabispidine compound bearing a N-(alkoxycarbonylamino)alkyl substituent from a mixture comprising said oxabispidine and a corresponding compound lacking such a substituent.

We have now surprisingly found that such salts may, when dispersed in an aqueous solvent system containing certain sulfonate anions, be readily and efficiently isolated from such mixtures.

### DISCLOSURE OF THE INVENTION

According to a first aspect of the invention, there is provided a process for isolating a salt of formula I,

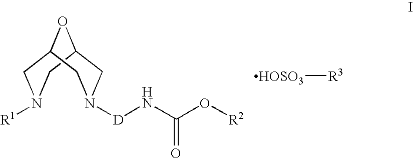

or a solvate thereof,
wherein R¹ represents H, an amino protective group or a structural fragment of formula Ia,

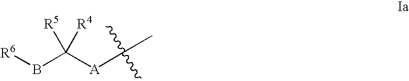

in which
R⁴ represents H, halo, C₁₋₆ alkyl, —OR⁷, -E-N(R⁸)R⁹ or, together with R⁵, represents ═O;
R⁵ represents H, C₁₋₆ alkyl or, together with R⁴, represents ═O;
R⁷ represents H, C₁₋₆ alkyl, -E-aryl, -E-Het¹, —C(O)R^{10a}, —C(O)OR^{10b} or —C(O)N(R^{11a})R^{11b};
R⁸ represents H, C₁₋₆ alkyl, -E-aryl, -E-Het¹, —C(O)R^{10a}, —C(O)OR^{10b}, S(O)₂R^{10c}, —[C(O)]ₚN(R^{11a})R^{11b} or —C(NH)NH₂;
R⁹ represents H, C₁₋₆ alkyl, -E-aryl or —C(O)R^{10d};
R^{10a} to R^{10d} independently represent, at each occurrence when used herein, C₁₋₆ alkyl (optionally substituted by one or more substituents selected from halo, aryl and Het²), aryl, Het³, or R^{10a} and R^{10d} independently represent H;
R^{11a} and R^{11b} independently represent, at each occurrence when used herein, H or C₁₋₆ alkyl (optionally substituted by one or more substituents selected from halo, aryl and Het⁴), aryl, Het⁵, or together represent C₃₋₆ alkylene, optionally interrupted by an O atom;
E represents, at each occurrence when used herein, a direct bond or C₁₋₄ alkylene;
p represents 1 or 2;

A represents a direct bond, -J-, -J-N(R^{12a})—, -J-S(O)₂N(R^{12b})—, -J-N(R^{12c})S(O)₂— or -J-O— (in which latter four groups, -J is attached to the oxabispidine ring nitrogen);

B represents -Z-{[C(O)]ₐC(H)(R^{13a})}_{b}—, -Z-[C(O)]_{c}N(R^{13b}), -Z-N(R^{13c})S(O)₂—, -Z-S(O)₂N(R^{13d})—, -Z-S(O)ₙ—, -Z-O— (in which latter six groups, Z is attached to the carbon atom bearing R⁴ and R⁵), —N(R^{13e})-Z-, —N(R^{13f})S(O)₂-Z-, —S(O)₂N(R³⁹)-Z- or —N(R^{13h})C(O)O-Z- (in which latter four groups, Z is attached to the R⁶ group);
J represents C₁₋₆ alkylene optionally interrupted by —S(O)₂N(R^{12d}) or —N(R^{12e})S(O)₂— and/or optionally substituted by one or more substituents selected from —OH, halo and amino;
Z represents a direct bond or C₁₋₄ alkylene, optionally interrupted by —N(R¹³ⁱ)S(O)₂— or —S(O)₂N(R^{13j})—;
a, b and c independently represent 0 or 1;
n represents 0, 1 or 2;
R^{12a} to R^{12e} independently represent, at each occurrence when used herein, H or C₁₋₆ alkyl;
R^{13a} represents H or, together with a single ortho-substituent on the R⁶ group (ortho-relative to the position at which the B group is attached), R^{13a} represents C₂₋₄ alkylene optionally interrupted or terminated by O, S, N(H) or N(C₁₋₆ alkyl);
R^{13b} represents H, C₁₋₆ alkyl or, together with a single ortho-substituent on the R⁶ group (ortho-relative to the position at which the B group is attached), R^{13b} represents C₂₋₄ alkylene;
R^{13c} to R^{13j} independently represent, at each occurrence when used herein, H or C₁₋₆ alkyl;
R⁶ represents phenyl or pyridyl, both of which groups are optionally substituted by one or more substituents selected from —OH, cyano, halo, nitro, C₁₋₆ alkyl (optionally terminated by —N(H)C(O)OR^{14a}), C₁₋₆ alkoxy, —N(R^{15a})R^{15b}, —C(O)R^{15c}, C(O)OR^{15d}, —C(O)N(R^{15e})R^{15f}, —N(R^{15g})C(O)R^{15h}, —N(R¹⁵ⁱ)C(O)N(R^{15j})R^{15k}, —N(R^{15m})S(O)R^{14b}, —S(O)₂N(R¹⁵ⁿ)R^{15o}, —S(O)₂R^{14c}, —OS(O)₂R^{14d} and/or aryl;
and an ortho-substituent (ortho-relative to the attachment of B) may
(i) together with R^{13a}, represent C₂₋₄ alkylene optionally interrupted or terminated by O, S, N(H) or N(C₁₋₆ alkyl), or
(ii) together with R^{13b}, represent C₂₋₄ alkylene;
   R^{14a} to R^{14d} independently represent C₁₋₆ alkyl;
   R^{15a} and R^{15b} independently represent H, C₁₋₆ alkyl or together represent C₃₋₆ alkylene, resulting in a four- to seven-membered nitrogen-containing ring;
   R^{15c} to R^{15o} independently represent H or C₁₋₆ alkyl; and
   Het¹ to Het⁵ independently represent, at each occurrence when used herein, five- to twelve-membered heterocyclic groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, —N(R^{16a})R^{16b}, —C(O)R^{16c}, —C(O)OR^{16d}, —C(O)N(R^{16e})R^{16f}, N(R^{16g})C(O)R^{16h}, —S(O)₂N(R¹⁶ⁱ)(R^{16j}) and/or —N(R^{16k})S(O)₂R^{16l};
   R^{16a} to R^{16l} independently represent C₁₋₆ alkyl, aryl or R^{16a} to R^{16k} independently represent H;
   provided that:
(a) when R⁵ represents H or C₁₋₆ alkyl; and
   A represents -J-N(R^{12a})— or -J-O—, then:
   (i) J does not represent C₁ alkylene or 1,1-C₂₋₆ alkylene; and
   (ii) B does not represent —N(R^{13b})—, —N(R^{13c})S(O)₂—, —S(O)ₙ—, —O—, —N(R^{13e})-Z, —N(R^{13f})S(O)₂-Z- or —N(R^{13h})C(O)O-Z-;
(b) when R⁴ represents —OR⁷ or -E-N(R⁸)R⁹ in which E represents a direct bond, then:
   (i) A does not represent a direct bond, -J-N(R^{12a})—, -J-S(O)₂—N(R^{12b})— or -J-O—; and
   (ii) B does not represent —N(R^{13b}), —N(R^{13c})S(O)₂—, —S(O)ₙ—, —O—, —N(R^{13e})-Z, —N(R^{13f})S(O)₂-Z- or —N(R^{13h})C(O)O-z-;
(c) when A represents -J-N(R^{12c})S(O)₂—, then J does not represent C₁ alkylene or 1,1-C₂₋₆ alkylene; and
(d) when R⁵ represents H or C₁₋₆ alkyl and A represents -J-S(O)₂N(R^{12b})—, then B does not represent —N(R^{13b})—, —N(R^{13c})S(O)₂—, —S(O)ₙ—, —O—, —N(R^{13e})-Z-, —N(R^{13f})S(O)₂-Z- or —N(R^{13h})C(O)O-Z-; and
   D represents optionally branched C₂₋₆ alkylene, provided that D does not represent 1,1-C₂₋₆ alkylene;
   R² represents C₁₋₆ alkyl (optionally substituted by one or more substituents selected from —OH, halo, cyano, nitro and aryl) or aryl; and
   R³ represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy;
   wherein each aryl and aryloxy group, unless otherwise specified, is optionally substituted;
   from a mixture comprising a compound of formula II,

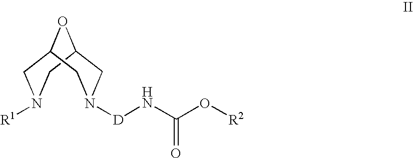

wherein D, R¹ and R² are as defined above, and a compound of formula III,

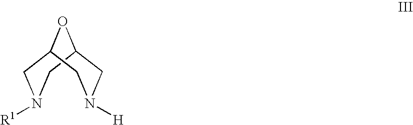

or a salt and/or a solvate thereof, wherein R¹ is as defined above;
which process comprises:
(1) providing, in an aqueous solvent system, a dispersion of
   (i) the compounds of formulae II and III, as defined above and
   (ii) a source of R³SO₃⁻ anions, wherein R³ is as defined above;
(2) if necessary, adjusting the pH of the aqueous dispersion to any value from 3 to 8; and
(3) isolating the solid salt of formula I, or solvate thereof, thereby formed,
   which process is hereinafter referred to as “the process of the invention”.

In a preferred embodiment of the process according to the first aspect of the invention, the compounds of formulae II and III are essentially the only compounds dispersed in the aqueous solvent system that comprise an oxabispidine structural unit. In this respect, it is preferred that, compared to the quantity of the compound of formula II present, the aqueous solvent system contains a total of no more that 0.1 (e.g. no more than 0.05, 0.04, 0.03 or, particularly, 0.025, 0.02, 0.015 or 0.01) molar equivalents of other oxabispidine-based compounds other than the compound of formula III.

When used herein with respect to salts of formula I, the term “isolation” includes references to obtaining the salt of formula I in a form that is substantially (e.g. 99% or, particularly, at least 99.5 or 99.8%) free of the compound of formula III or salt(s) thereof.

When used herein, the term “aqueous solvent system” includes references to water and mixtures of water and water-miscible organic solvents (e.g. di(C₁₋₄ alkyl)ethers (such as tetrahydrofuran), dioxane, acetonitrile, acetone and, particularly, C₁₋₄ alkyl alcohols such as methanol, ethanol, n-propanol, and isopropanol). The most preferred aqueous solvent systems are water and, particularly, mixtures of water and any of the above-mentioned alcohols (such as isopropanol). In this respect, preferred mixtures of water and C₁₋₄ alkyl alcohols (e.g. isopropanol) include those that comprise from 2 to 30% v/v (e.g. from 5 to 18% v/v) of the alcohol.

When used herein, the term “source of R³SO₃⁻ anions” includes references to any salt or compound that, on dispersion in water, dissociates (or is capable of dissociating) so as to provide cations and R³SO₃⁻ anions. In this respect, suitable sources of R³SO₃⁻ anions that may be mentioned include R³SO₃H and (R³SO₃)ₙM, wherein M is a metal of valency n, and n is an integer from 1 to 3. Preferred sources of R³SO₃⁻ are R³SO₃H or, particularly, R³SO₃M¹, wherein M¹ is an alkali metal such as sodium or potassium.

Unless otherwise specified, alkyl groups and alkoxy groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl and alkoxy groups may also be part cyclic/acyclic. Such alkyl and alkoxy groups may also be saturated or, when there is a sufficient number (i.e. a minimum of two) of carbon atoms, be unsaturated and/or interrupted by one or more oxygen and/or sulfur atoms. Unless otherwise specified, alkyl and alkoxy groups may also be substituted by one or more halo, and especially fluoro, atoms.

Unless otherwise specified, alkylene groups as defined herein may be straight-chain or, when there is a sufficient number (i.e. a minimum of two) of carbon atoms, be branched-chain. Such alkylene chains may also be saturated or, when there is a sufficient number (i.e. a minimum of two) of carbon atoms, be unsaturated and/or interrupted by one or more oxygen and/or sulfur atoms. Unless otherwise specified, alkylene groups may also be substituted by one or more halo atoms.

The term “aryl”, when used herein, includes C₆₋₁₃ aryl (e.g. C₆₋₁₀) groups. Such groups may be monocyclic, bicyclic or tricylic and, when polycyclic, be either wholly or partly aromatic. In this respect, C₆₋₁₃ aryl groups that may be mentioned include phenyl, naphthyl, 1,2,3,4-tetrahydronaphthyl, indanyl, indenyl, fluorenyl and the like. For the avoidance of doubt, the point of attachment of substituents on aryl groups may be via any carbon atom of the ring system.

Similarly, the term “aryloxy”, when used herein includes C₆₋₁₃ aryloxy groups such as phenoxy, naphthoxy, fluorenoxy and the like. For the avoidance of doubt, aryloxy groups referred to herein are attached to the rest of the molecule via the O-atom of the oxy-group.

Unless otherwise specified, aryl and aryloxy groups may be substituted by one or more substituents selected from —OH, cyano, halo, nitro, C₁₋₆ alkyl, C₁₋₆ alkoxy, N(R^{15a})R^{15b}, —C(O)R^{15c}, —C(O)OR^{15d}, —C(O)N(R^{15e})R^{15f}, N(R^{15g})C(O)R^{15h}, —N(R^{15m})S(O)₂R^{14b}, —S(O)₂N(R¹⁵ⁿ)(R^{15o}), —S(O)₂R^{14c} and/or —OS(O)₂R^{14d}, (wherein R^{14b} to R^{14d} and R^{15a} to R^{15o} are as hereinbefore defined). When substituted, aryl and aryloxy groups are preferably substituted by between one and three substituents. For the avoidance of doubt, the point of attachment of substituents on aryl groups may be via any carbon atom of the ring system.

The term “halo”, when used herein, includes fluoro, chloro, bromo and iodo.

Het (Het¹, Het², Het³, Het⁴ and Het⁵) groups that may be mentioned include those containing 1 to 4 heteroatoms (selected from the group oxygen, nitrogen and/or sulfur) and in which the total number of atoms in the ring system are between five and twelve. Het (Het¹, Het², Het³, Het⁴ and Het⁵) groups may be fully saturated, wholly aromatic, partly aromatic and/or bicyclic in character. Heterocyclic groups that may be mentioned include 1-azabicyclo[2.2.2]octanyl, benzimidazolyl, benzisoxazolyl, benzodioxanyl, benzodioxepanyl, benzodioxolyl, benzofuranyl, benzofurazanyl, benzomorpholinyl, 2,1,3-benzoxadiazolyl, benzoxazinonyl, benzoxazol-idinyl, benzoxazolyl, benzopyrazolyl, benzo[e]pyrimidine, 2,1,3-benzothiadiazolyl, benzothiazolyl, benzothienyl, benzotriazolyl, chromanyl, chromenyl, cinnolinyl, 2,3-dihydrobenzimidazolyl, 2,3-dihydrobenzo[b]furanyl, 1,3-dihydrobenzo[c]furanyl, 2,3-dihydropyrrolo[2,3-b]pyridyl, dioxanyl, furanyl, hexahydropyrimidinyl, hydantoinyl, imidazolyl, imidazo[1,2-a]pyridyl, imidazo-[2,3-b]thiazolyl, indolyl, isoquinolinyl, isoxazolyl, maleimido, morpholinyl, oxadiazolyl, 1,3-oxazinanyl, oxazolyl, phthalazinyl, piperazinyl, piperidinyl, purinyl, pyranyl, pyrazinyl, pyrazolyl, pyridyl, pyrimidinyl, pyrrolidinonyl, pyrrolidinyl, pyrrolinyl, pyrrolo[2,3-b]pyridyl, pyrrolo[5,1-b]pyridyl, pyrrolo[2,3-c]pyridyl, pyrrolyl, quinazolinyl, quinolinyl, sulfolanyl, 3-sulfolenyl, 4,5,6,7-tetrahydrobenzimidazolyl, 4,5,6,7-tetrahydrobenzopyrazolyl, 5,6,7,8-tetra-hydrobenzo[e]pyrimidine, tetrahydrofuranyl, tetrahydropyranyl, 3,4,5,6-tetra-hydropyridyl, 1,2,3,4-tetrahydropyrimidinyl, 3,4,5,6-tetrahydropyrimidinyl, thiadiazolyl, thiazolidinyl, thiazolyl, thienyl, thieno[5,1-c]pyridyl, thiochromanyl, triazolyl, 1,3,4-triazolo[2,3-b]pyrimidinyl and the like.

Substituents on Het (Het¹, Het², Het³, Het⁴ and Het⁵) groups may, where appropriate, be located on any atom in the ring system including a heteroatom. The point of attachment of Het (Het¹, Het², Het³, Het⁴ and Het⁵) groups may be via any atom in the ring system including (where appropriate) a heteroatom, or an atom on any fused carbocyclic ring that may be present as part of the ring system. Het (Het¹, Het², Het³, Het⁴ and Het⁵) groups may also be in the N- or S-oxidised form.

Solvates of the salt of formula I that may be mentioned include hydrates, such as monohydrates or hemi-hydrates.

Compounds employed in or produced by the process of the invention may exhibit tautomerism. The process of the invention encompasses the use or production of such compounds in any of their tautomeric forms, or in mixtures of any such forms.

Similarly, the compounds employed in or produced by the process of the invention may also contain one or more asymmetric carbon atoms and may therefore exist as enantiomers or diastereoisomers, and may exhibit optical activity. The process of the invention thus encompasses the use or production of such compounds in any of their optical or diastereoisomeric forms, or in mixtures of any such forms.

Abbreviations are listed at the end of this specification.

As used herein, the term “amino protective group” includes groups mentioned in “Protective Groups in Organic Synthesis”, 3^{rd} edition, T. W. Greene & P. G. M. Wutz, Wiley-Interscience (1999), in particular those mentioned in the chapter entitled “Protection for the Amino Group” (see pages 494 to 502) of that reference, the disclosure in which document is hereby incorporated by reference.

Specific examples of amino protective groups thus include:
(a) those which form carbamate groups (e.g. to provide methyl, cyclopropylmethyl, 1-methyl-1-cyclopropylmethyl, diisopropylmethyl, 9-fluorenylmethyl, 9-(2-sulfo)fluorenylmethyl, 2-furanylmethyl, 2,2,2-trichloroethyl, 2-haloethyl, 2-trimethylsilylethyl, 2-methylthioethyl, 2-methylsulfonylethyl, 2(p-toluenesulfonyl)ethyl, 2-phosphonioethyl, 1,1-dimethylpropynyl, 1,1-dimethyl-3-(N,N-dimethylcarboxamido)propyl, 1,1-dimethyl-3-(N,N-diethylamino)-propyl, 1-methyl-1-(1-adamantyl)ethyl, 1-methyl-1-phenylethyl, 1-methyl-1-(3,5-dimethoxyphenyl)ethyl, 1-methyl-1-(4-biphenylyl)ethyl, 1-methyl-1-(p-phenylazophenyl)ethyl, 1,1-dimethyl-2-haloethyl, 1,1-dimethyl-2,2,2-trichloroethyl, 1,1-dimethyl-2-cyanoethyl, isobutyl, t-butyl, t-amyl, cyclobutyl, 1-methylcyclobutyl, cyclopentyl, cyclohexyl, 1-methylcyclohexyl, 1-adamantyl, isobornyl, vinyl, allyl, cinnamyl, phenyl, 2,4,6-tri-t-butylphenyl, m-nitrophenyl, S-phenyl, 8-quinolinyl, N-hydroxypiperidinyl, 4-(1,4-dimethylpiperidinyl), 4,5-diphenyl-3-oxazolin-2-one, benzyl, 2,4,6-trimethylbenzyl, p-methoxy-benzyl, 3,5-dimethoxybenzyl, p-decyloxybenzyl, p-nitrobenzyl, o-nitro-benzyl, 3,4-dimethoxy-6-nitrobenzyl, p-bromobenzyl, chlorobenzyl, 2,4-dichlorobenzyl, p-cyanobenzyl, o-(N,N-dimethylcarboxamidobenzyl)-benzyl, m-chloro-p-acyloxybenzyl, p-(dihydroxyboryl)benzyl, p-(phenyl-azo)benzyl, p-(p′-methoxyphenylazo)benzyl, 5-benzisoxazolylmethyl, 9-anthrylmethyl, diphenylmethyl, phenyl(o-nitrophenyl)methyl, di(2-pyridyl)methyl, 1-methyl-1-(4-pyridyl)-ethyl, isonicotinyl, or S-benzyl, carbamate groups);
(b) those which form amide groups (e.g. to provide N-formyl, N-acetyl, N-chloroacetyl, N-dichloroacetyl, N-trichloroacetyl, N-trifluoroacetyl, N-o-nitrophenylacetyl, N-o-nitrophenoxyacetyl, N-acetoacetyl, N-acetyl-pyridinium, N-3-phenylpropionyl, N-3-(p-hydroxyphenyl)propionyl, N-3-(o-nitrophenyl)propionyl, N-2-methyl-2-(o-nitrophenoxy)propionyl, N-2-methyl-2-(o-phenylazophenoxy)propionyl, N-4-chlorobutyryl, N-isobutyryl, N-o-nitrocinnamoyl, N-picolinoyl, N—(N′-acetylmethionyl), N—(N′-benzoylphenylalanyl), N-benzoyl, N-p-phenylbenzoyl, N-p-methoxybenzoyl, N-o-nitrobenzoyl, or N-o-(benzoyloxymethyl)benzoyl, amide groups);
(c) those which form N-alkyl groups (e.g. N-allyl, N-phenacyl, N-3-acetoxypropyl, N-(4-nitro-1-cyclohexyl-2-oxo-pyrrolin-3-yl), N-methoxy-methyl, N-chloroethoxymethyl, N-benzyloxymethyl, N-pivaloyloxymethyl, N-2-tetrahydropyranyl, N-2,4-dinitrophenyl, N-benzyl, N-3,4-di-methoxy-benzyl, N-o-nitrobenzyl, N-di(p-methoxyphenyl)methyl, N-triphenylmethyl, N-(p-methoxyphenyl)-diphenylmethyl, N-diphenyl-4-pyridylmethyl, N-2-picolyl N′-oxide, or N-dibenzosuberyl, groups);
(d) those which form N-phosphinyl and N-phosphoryl groups (e.g. N-diphenylphosphinyl, N-dimethylthiophosphinyl, N-diphenylthiophosphinyl, N-diethyl-phosphoryl, N-dibenzylphosphoryl, or N-phenylphosphoryl, groups);
(e) those which form N-sulfenyl groups (e.g. N-benzenesulfenyl, N-o-nitro-benzenesulfenyl, N-2,4-dinitrobenzenesulfenyl, N-pentachlorobenzene-sulfenyl, N-2-nitro-4-methoxybenzenesulfenyl, or N-triphenylmethyl-sulfenyl, groups);
(f) those which form N-sulfonyl groups (e.g. N-benzenesulfonyl, N-p-nitrobenzenesulfonyl, N-p-methoxybenzenesulfonyl, N-2,4,6-trimethyl-benzenesulfonyl, N-toluenesulfonyl, N-benzylsulfonyl, N-p-methylbenzyl-sulfonyl, N-trifluoromethylsulfonyl, or N-phenacylsulfonyl, groups); and
(g) that which forms the N-trimethylsilyl group.

Preferred amino protective groups include those which provide the carbamate, N-alkyl and N-sulfonyl groups mentioned above. Particular protecting groups thus include tert-butoxycarbonyl (to form a tert-butylcarbamate group), benzenesulfonyl, 4-nitrobenzenesulfonyl and optionally substituted benzyl groups, such as 3,4-dimethoxybenzyl, o-nitrobenzyl, (benzyl)benzyl (e.g. (4-benzyl)-benzyl) and, especially, unsubstituted benzyl groups.

Preferred values of R¹ include an amino protecting group or a structural fragment of formula Ia in which:

R⁴ represents H, halo, C₁₋₃ alkyl, —OR⁷, —N(H)R⁸ or, together with R⁵, represents ═O;
R⁵ represent H, C₁₋₃ alkyl or, together with R⁴, represents ═O;
R⁷ represents H, C₁₋₆ alkyl, -E-(optionally substituted phenyl) or -E-Het¹;
R⁸ represents H, C₁₋₆ alkyl, -E-(optionally substituted phenyl), —C(O)R^{10a}, C(O)OR^{10b}, S(O)₂R^{10c}, —C(O)N(R^{11a})R^{11b} or —C(NH)NH₂;
R^{10a} to R^{10c} independently represent C₁₋₆ alkyl, or R^{10a} represents H;
R^{11a} and R^{11b} independently represent H or C₁₋₄ alkyl;
E represents, at each occurrence when used herein, a direct bond or C₁₋₂ alkylene;
A represents -J-, -J-N(R^{12a})— or -J-O—;
B represents -Z-, -Z-N(R^{13b})—, -Z-S(O)ₙ— or -Z-O—;
J represents C₁₋₄ alkylene;
Z represents a direct bond or C₁₋₃ alkylene;
R^{12a} and R^{13b} independently represent H or C₁₋₄ alkyl;
n represents 0 or 2;
R⁶ represents phenyl or pyridyl, both of which groups are optionally substituted by one or more substituents selected from cyano, halo, nitro, C₁₋₆ alkyl, C₁₋₆ alkoxy, —NH₂, —C(O)N(R^{15e})R^{15f}, —N(R^{15g})C(O)R^{15h} and —N(R^{15m})S(O)₂—R^{14b};
R^{14b} represents C₁₋₃ alkyl;
R^{15e} to R^{15m} independently represent, at each occurrence when used herein, H or C₁₋₄ alkyl;
Het¹ to Het⁵ are optionally substituted by one or more substituents selected from ═O, cyano, halo, nitro, C₁₋₄ alkyl, C₁₋₄ alkoxy, —N(R^{16a})R^{16b}, —C(O)R^{16c} and C(O)OR^{16d};
R^{16a} to R^{16d} independently represent H, C₁₋₄ alkyl or aryl;
optional substituents on aryl and aryloxy groups, are unless otherwise stated, one or more substituents selected from cyano, halo, nitro, C₁₋₄ alkyl and C₁₋₄ alkoxy.

Values of R¹ that are more preferred include an amino protective group, or a structural fragment of formula Ia in which:

R⁴ represents H, methyl, —OR⁷ or —N(H)R⁸;
R⁵ represents H or methyl;
R⁷ represents H, C₁₋₂ alkyl or phenyl (which phenyl group is optionally substituted by one or more substituents selected from cyano and C₁₋₄ alkoxy);
R⁸ represents H, C₁₋₂ alkyl, phenyl (which phenyl group is optionally substituted by one or more substituents selected from cyano, halo, nitro, C₁₋₄ alkyl and C₁₋₄ alkoxy), —C(O)—R^{10a} or —C(O)O—R^{10b};
R^{10a} and R^{10b} independently represent C₁₋₆ alkyl;
A represents C₁₋₄ alkylene;
B represents -Z-, -Z-N(R^{13b})—, -Z-S(O)₂— or -Z-O—;
R^{13b} represents H or methyl;
R⁶ represents pyridyl or phenyl, which latter group is optionally substituted by one to three substituents selected from halo or, particularly, cyano, nitro, C₁₋₂ alkoxy, NH₂ and —N(H)S(O)₂CH₃.

Values of R¹ that are more preferred still include an amino protective group, or a structural fragment of formula Ia in which:

R⁴ represents H, —OR⁷ or —N(H)R⁸;
R⁷ represents H or phenyl (optionally substituted by one or more substituents selected from cyano and C₁₋₂ alkoxy);
R⁸ represents H, phenyl (optionally substituted by one or more cyano groups) or —C(O)O—C₁₋₅ alkyl;
A represents C₁₋₃ alkylene;
B represents -Z-, -Z-N(H)—, -Z-S(O)₂— or -Z-O—;
R⁶ represents phenyl substituted by cyano in the para-position (relative to B) and optionally substituted by fluoro in the ortho-position (relative to B) (e.g. phenyl substituted by cyano in the ortho- and/or, in particular, the para-position relative to B).

Particularly preferred values of R¹ include an amino protective group, or a structural fragment of formula Ia in which:

R⁴ represents H or —OH;
R⁵ represents H;
A represents CH₂;
B represents -Z-, -Z-N(H)— or -Z-O;
Z represents a direct bond or C₁₋₂ alkylene;
R⁶ represents 2-fluoro-4-cyanophenyl or, particularly, para-cyanophenyl.

Especially preferred values of R¹ include an include an amino protective group, or the following sub-structures

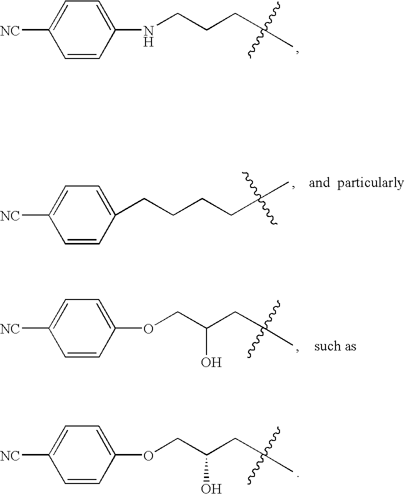

In an alternative embodiment of the invention, values of R¹ that may be mentioned include the following sub-structures

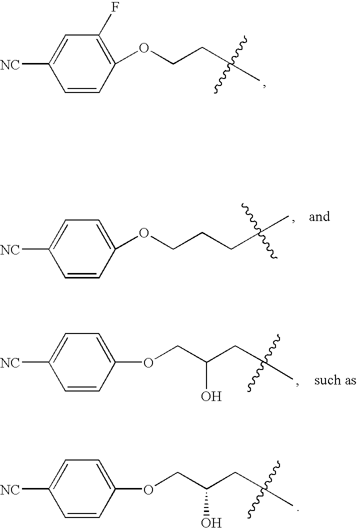

The process of the invention is most preferably carried out to provide salts of formula I in which R¹ is an amino protective group as defined above, such as benzyl.

Preferred values of D include —(CH₂)₃— or, particularly, —(CH₂)₂—.

Preferred values of R² include C₁₋₆ alkyl, particularly saturated C₁₋₆ alkyl.

More preferred values of R² include saturated C₃₋₅ alkyl, particularly saturated C₄ alkyl, such as tert-butyl.

Preferred values of R³ include phenyl, optionally substituted by one or more (e.g. one to three) substituents (e.g. one substituent) selected from C₁₋₃ alkyl (e.g. methyl), halo and nitro, particularly unsubstituted phenyl, methylphenyl (such as 4-methylphenyl) or trimethylphenyl (such as 2,4,6-trimethylphenyl).

The most preferred value of R³ is 2,4,6-trimethylphenyl.

In an alternative embodiment of the invention (e.g. when D represents —(CH₂)₃—), R³ represents 4-halophenyl (e.g. 4-chlorophenyl).

Thus, particularly preferred salts of formula I include salts of formula Ib,

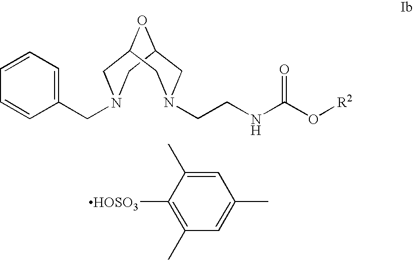

or a hydrate thereof
wherein R² is as defined above.

In an alternative embodiment of the invention, other salts of formula I that may be mentioned include salts of formula Ic,

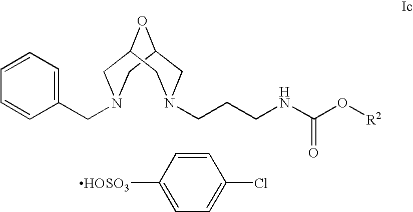

or a hydrate thereof
wherein R² is as defined above.

It is preferred that the molar quantity of R³SO₃⁻ anions is approximately equal to the molar quantity of the compound of formula II. In this respect, the molar ratio of R³SO₃⁻ anions to compound of formula II is preferably any value from 15:10 to 10:15, such as from 12:10 to 10:11 (e.g. about 1:1).

When adjustment of the pH of the aqueous mixture takes place (step (2) above), the pH to which the mixture is adjusted is preferably any value from 4 to 7 (e.g. from 5 to 7).

If the pH of the aqueous mixture is adjusted, a weak, water-soluble acid is preferably employed to effect the adjustment. The term “weak, water-soluble acid”, when used herein, includes references to acids that have a solubility in water of 1 mg/mL or more and a pKa (measured in water) of any value from 2 to 7 (preferably from 3 to 5). In this respect, preferred weak, water-soluble acids that may be mentioned include carboxylic acids such as acetic or, particularly, citric acid.

The salt of formula I, or solvate thereof, may be isolated by methods known to those skilled in the art, such as those described hereinafter (e.g. filtration).

In a preferred embodiment of the first aspect of the invention, the mixture of compounds of formulae II and III is obtained by incomplete reaction of a compound of formula III, as hereinbefore defined, or a salt and/or solvate thereof, with a compound of formula IV,

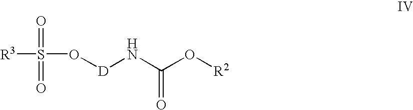

wherein D, R² and R³ are as hereinbefore defined, in the presence of solvent and base.

Suitable bases for the reaction between the compounds of formulae III and IV include water-soluble bases such as alkali metal hydroxides, alkali metal carbonates and/or alkali metal hydrogencarbonates. Particularly preferred bases include alkali metal hydroxides, such as potassium hydroxide or, particularly, sodium hydroxide.

The skilled person will appreciate that R³SO₃⁻ anions are a by-product of the reaction between the compounds of formulae III and IV (i.e. they are produced by way of a nucleophilic displacement from the compound of formula IV).

It is possible for these anions to be utilised in step (1) of the process according to the first aspect of the invention. Thus, when the mixture of compounds of formulae II and III is obtained by incomplete reaction of a compound of formula III, as hereinbefore defined, or a salt and/or solvate thereof, with a compound of formula IV, it is preferred that R³SO₃⁻ anions present in the aqueous dispersion of step (1) above are derived from the compound of formula IV.

By “derived from the compound of formula IV”, we mean that the R³SO₃⁻ anions of step (1) above are, either wholly or in part, obtained (via nucleophilic displacement of R³SO₃⁻ from the compound of formula IV) through reaction between the compounds of formulae III and IV. It is particularly preferred that substantially all (e.g. greater than 95%) of the R³SO₃⁻ anions utilised in step (1) above are derived from the compound of formula IV in this way.

One way of obtaining the ReSO₃⁻ anions derived from the compound of formula IV in a convenient form for use in step (1) of the process according to the first aspect of the invention is to utilise base and an aqueous solvent system in the reaction between the compounds of formulae III and IV. In this way, the R³SO₃-anions, once formed, can be made to disperse into the aqueous solvent system.

Thus, in a particularly preferred embodiment of the first aspect of the invention, the mixture of compounds of formulae II and III is obtained by incomplete reaction of the compounds of formulae III and IV in the presence of an aqueous phase and base.

When used herein, the term “in the presence of an aqueous phase” includes references to reactions conducted in the presence of a solvent system that is:
(a) monophasic and based upon (e.g. consisting essentially of) an aqueous solvent system, i.e. forming a monophasic aqueous solvent system; or
(b) part-aqueous and biphasic, i.e. forming a biphasic system consisting of two immiscible phases, one that is based upon (e.g. consisting essentially of) an aqueous solvent system and another that is based upon (e.g. consisting essentially of) an organic solvent system.

When used herein, the term “organic solvent system” includes references to a single organic solvent as well as to mixtures of two or more organic solvents. Organic solvents that may be mentioned in this respect include: di(C₁₋₆ alkyl)ethers (such as di(C₁₋₄ alkyl)ethers, e.g. diethyl ether); C₁₋₆ alkyl acetates (such as C₁₋₄ alkyl acetates, e.g. ethyl acetate); chlorinated hydrocarbons (e.g. chlorinated C₁₋₄ alkanes such as dichloromethane, chloroform and carbon tetrachloride); hexane; petroleum ether: aromatic hydrocarbons, such as benzene and mono-, di- or tri-alkylbenzenes (e.g. mesitylene, xylene, or toluene); and mixtures thereof. Preferred organic solvent systems include benzene or, particularly, toluene.

When conducted in a monophasic aqueous solvent system, incomplete reaction between the compounds of formulae III and IV may directly provide, dispersed in the aqueous solvent system, a mixture of the compounds of formula II and III, as well as a source of R³SO₃⁻ anions (through nucleophilic displacement of sulfonate from the compound of formula IV).

Thus, according to a second aspect of the invention, there is provided a process for preparing a salt of formula I, as hereinbefore defined, or solvate thereof, which process comprises:
(I) effecting reaction between base, a compound of formula III, as hereinbefore defined, or a salt and/or solvate thereof and a compound of formula IV, as hereinbefore defined, in the presence of a monophasic aqueous solvent system;
(II) if necessary, adjusting the pH of the resulting aqueous dispersion to any value from 3 to 8; and
(III) isolating the solid salt of formula I, or solvate thereof, thereby formed.

In this aspect of the invention, preferences for the salts of formula I, base and pH adjustment are the same as those set out above with respect to the first aspect of the invention.

It is preferred that step (I) above comprises effecting incomplete reaction between base, a compound of formula III, as hereinbefore defined, or a salt and/or solvate thereof and a compound of formula IV, as hereinbefore defined, in the presence of a monophasic aqueous solvent system.

After step (I) above, and either before or after step (II) above, a water-miscible alcohol (for example an alcohol such as one of those mentioned above with respect to water-miscible organic solvents (e.g. isopropanol)) is optionally added to the reaction mixture, so as to facilitate a controlled precipitation of the salt of formula I. The water-miscible alcohol may be added regardless of whether or not the aqueous solvent system employed in step (I) includes a C₁₋₄ alkyl alcohol, but, if employed, is preferably added in such an amount that water-miscible alcohol(s) represent(s) from 2 to 30% v/v (e.g. from 5 to 18% v/v) of the resulting solvent system.

When the reaction between the compounds of formulae III and IV is conducted in the presence of base and a solvent system that is part-aqueous and biphasic, the resulting mixture of compounds of formulae II and III may reside in a different phase (e.g. the organic phase) to the source of R³SO₃⁻ anions (which will typically reside in the aqueous phase). Thus, to provide the aqueous dispersion set out in step (1) of the process according to the first aspect of the invention, it is convenient, in these circumstances, to extract the compounds of formulae II and III into an aqueous solvent system.

Thus, according to a third aspect of the invention, there is provided a process for preparing a salt of formula I, as hereinbefore defined, or a solvate thereof, which process comprises:
(A) effecting reaction between base, a compound of formula III, as hereinbefore defined, or a salt and/or solvate thereof and a compound of formula IV, as hereinbefore defined, in the presence of base and a solvent system that is part-aqueous and biphasic;
(B) separating the first organic and first aqueous phases that are obtained after performance of step (A), and retaining both of these phases;
(C) extracting the first organic phase with an aqueous solution of an acid to produce a second aqueous phase;
(D) separating the second aqueous phase and then combining it with the first aqueous phase to produce a precipitation mixture;
(E) if necessary, adjusting the pH of the precipitation mixture to any value from 3 to 8; and then
(F) isolating the solid salt of formula I, or solvate thereof, thereby formed.

In this aspect of the invention also, preferences for the salts of formula I, base and pH adjustment are the same as those set out above with respect to the first aspect of the invention.

Again, it is preferred that step (A) above comprises effecting incomplete reaction between base, a compound of formula III, as hereinbefore defined, or a salt and/or solvate thereof and a compound of formula IV, as hereinbefore defined, in the presence of base and a solvent system that is part-aqueous and biphasic.

When used herein, the term “effecting incomplete reaction” includes references to effecting reaction to anywhere from 75 to 99.9% (e.g. from 90 to 99.9% completion, such as from 95 to 99%) completion. For the avoidance of doubt, percentage completion is calculated by reference to the consumption of the reagent having the lowest number of molar equivalents present in the reaction mixture (which may, in certain embodiments, be the compound of formula III, or salt and/or solvate thereof). Further, reaction between the compounds of formulae III and IV is effected so as to provide a compound of formula II (or, depending upon the conditions employed, salt of formula I).

For the avoidance of doubt, the solvent system that is part-aqueous and biphasic (i.e. that employed in step (A) above) comprises two separate, immiscible phases, one consisting essentially of an aqueous solvent system, as defined above, and the other consisting of an organic solvent system, as also defined above. Preferred aqueous solvent systems that may be utilised in this aspect of the invention include water.

Base may be employed in step (A) as a solid, or, preferably, in the form of an aqueous solution. When base is employed as an aqueous solution, the molarity of the solution is in the range 1 to 5 M, for example 2 to 4 M, and preferably between 2.25 and 3.5 M such as about 2.5 M. When such an aqueous solution is employed, this may constitute a part or, preferably, the whole of the aqueous phase of the solvent system of step (A) above (i.e. the solvent system that is part-aqueous and biphasic).

Base may be added in step (A) to the compound of formula III prior to, at the same time as, or after the addition of the compound of formula IV. When added after the addition of the compound of formula IV, the base may be added substantially in one portion or over any period of time from 30 minutes to 8 hours, such as from 3 hours to 6 hours. Preferably, the base is added substantially in one portion prior to the addition of the compound of formula IV.

The quantity of base employed is preferably sufficient to neutralise the sulfonic acid created by reaction between the compounds of formulae III and IV (e.g. an amount that is at least equimolar to the quantity of the compound of formula III employed). Further, if the compound of formula III is present in salt form, the quantity of base employed should also be sufficient to liberate the free base form of the compound of formula III (e.g. if a diprotonated salt of formula III is employed, then the quantity of base used is preferably at least three molar equivalents compared to the amount of the salt of formula III).

When a dihydrohalide (e.g. dihydrochloride) salt of a compound of formula III is employed, then the stoichiometric ratio of the compound of formula III to base is preferably in the range from 1:2 to 1:5, particularly in the range from 1:3 to 1:4 such as from 10:32 to 10:33 or thereabouts.

The organic solvent component of the biphasic solvent system of step (A) above may be added to the compound of formula III prior to, at the same time as, or after the addition of the compound of formula IV.

The compound of formula IV may be added to the reaction mixture of step (A) above as a solid. In this instance, the organic solvent of the biphasic solvent system may be added to the reaction mixture before, during or after (e.g. either before or after) the addition of the compound of formula IV. Alternatively, the compound of formula IV may be added in the form of a solution, e.g. dissolved in an organic solvent which then forms the whole or, preferably, part of the organic phase of the biphasic solvent system. In this instance, the compound of formula IV may be mixed with the organic solvent in a separate vessel and the resulting mixture may be warmed (e.g. to any temperature from 28 to 40° C.) to promote dissolution of the compound of formula IV.

The reaction between the compounds of formulae III and IV (i.e. step (A) above) may be carried out at, or above, ambient temperature (e.g. at any temperature from 10 to 100° C., preferably from 25 to 90° C., and particularly from 50 to 80° C.). For example, when the solvent system that is employed is a mixture of water and toluene, the reaction may be carried out at any temperature from 55 to 75° C. (such as from 60 to 70° C.).

The reaction mixture may be stirred at the specified temperature for any period of time, such as from 1 hour to 24 hours, for example from 4 to 16 hours, depending upon, inter alia, the concentration of reagents and reaction temperature employed. The skilled person will appreciate that the temperature of the reaction will affect the time for the completion of step (a). For example, conducting the reaction at a lower temperature may require a longer reaction time than that necessary if the reaction is conducted at a higher temperature (and vice versa).

The stoichiometric ratio of the compound of formula III to the compound of formula IV is preferably in the range 3:2 to 2:3, particularly in the range 1:1 to 4:5 such as 20:21.

For step (B) above, the separation of the first organic phase from the first aqueous phase is, preferably, conducted at the same temperature as the reaction between compounds of formulae III and IV (i.e. step (A)—see above).

It is preferred that the acid employed in step (C) above is a weak, water soluble acid, such as one hereinbefore defined in respect of the first aspect of the invention.

The quantity of acid employed in step (C) above is preferably sufficient to extract into the second aqueous phase substantially all compound of formula II and compound of formula III that is present in the first organic phase. The stoichiometric ratio of the compound of formula III (the amount utilised in step (A) above) to acid, when the acid is triprotic (e.g. citric acid), is therefore preferably any value from 2:1 to 1:3 (e.g. from 18:10 to 10:25, such as from 17:10 to 12:10).

In the processes according to the first to third aspects of the invention, solvates of the compound of formula III that may be mentioned include hydrates. Salts of the compound of formula III that may be mentioned include acid addition salts, such as mono- or di-hydrohalides (e.g. dihydrochlorides). Solvates of the salts of the compounds of formula III may be mentioned include hydrates such as mono- or, particularly, hemi-hydrates.

Unless otherwise stated, when molar equivalents and stoichiometric ratios are quoted herein with respect to acids and bases, these assume the use of acids and bases that provide or accept only one mole of hydrogen ions per mole of acid or base, respectively. The use of acids and bases having the ability to donate or accept more than one mole of hydrogen ions is contemplated and requires corresponding recalculation of the quoted molar equivalents and stoichiometric ratios. Thus, for example, where the acid employed is diprotic, then only half the molar equivalents will be required compared to when a monoprotic acid is employed. Similarly, the use of a dibasic compound (e.g. Na₂CO₃) requires only half the molar quantity of base to be employed compared to what is necessary where a monobasic compound (e.g. NaHCO₃) is used, and so on.

The extraction of step (C) may be performed at, or above, ambient temperature, preferably at any temperature from room temperature to 75° C., particularly from 30 to 60° C., such as at 40° C. or thereabouts.

Preferably, when the first aqueous phase and the second aqueous phase are combined (step (D) above), additional water and/or a water-miscible alcohol (e.g. an alcohol such as one of those mentioned above with respect to water-miscible organic solvents) is added so that it is present in the resulting precipitation mixture.

Preferred water-miscible alcohols include methanol, ethanol, n-propanol and, particularly, isopropanol. The water-miscible alcohol is preferably present in the resulting precipitation mixture in an amount from 2 to 30% v/v (e.g. from 5 to 18% v/v).

The additional water and/or the water-miscible alcohol are preferably added to the first aqueous phase before that phase is combined with the second aqueous phase. In an alternative embodiment of the invention, and when both water and water-miscible alcohol are added to the first aqueous phase, the charge of water is added before or during the reaction between the compounds of formulae III and IV, and the charge of water-miscible alcohol is added to the first aqueous phase only after that phase has been separated from the first organic phase (i.e. after step (B) above).

Also, it is preferred that the first and second aqueous phases are combined at elevated temperature (e.g. at above 50° C., such as at any temperature from 60 to 80° C. (e.g. from 70 to 80° C., or at 65 or 75° C.). Preferably, the second aqueous phase is added to the first aqueous phase. When the two aqueous phases are combined at elevated temperature, it is preferred that the first aqueous phase is heated to that elevated temperature, after which the second aqueous phase is added at such a rate as to substantially maintain that elevated temperature. When the first and second aqueous phases have been combined, the elevated temperature (if employed in the combination process) may be maintained for any length of time, such as from 10 minutes to 2 hours, preferably for about 1 hour.

When adjustment of the pH takes place (i.e. step (E) above), the pH is adjusted as hereinbefore described in respect of the first aspect of the invention.

The solid salt of formula I isolated in step (F) above is formed by allowing the precipitation mixture to stand and/or, if elevated temperature is employed when combining the first and second aqueous phases, by cooling the precipitation mixture to ambient temperature or below (e.g. to any temperature from 0 to 30° C., such as from 5 to 25° C.). In such instances, the precipitation mixture is cooled or allowed to cool for any length of time, such as from 30 minutes to 12 hours, preferably from 2 to 6 hours, such as 4 hours or thereabouts.

The isolation of step (F) may be performed using known techniques, such as by filtration and/or evaporation of solvents, for example as described hereinafter.

The salt of formula I may, if desired, be further purified by recrystallisation from a suitable solvent system, such as water and/or a water-miscible lower (e.g. C₁₋₆) alkyl alcohol, preferably a C₁₋₄ alkyl alcohol, for example an optionally branched propyl alcohol, such as isopropanol.

Alternatively, purification may be effected by washing the salt of formula I with solvents, such as those mentioned hereinbefore with respect to recrystallisation.

In the second aspect of the invention (preparation of a salt of formula I via reaction between compounds of formulae III and IV in the presence of a monophasic aqueous solvent system), the base and compounds of formulae III and IV may be combined in any order. Further, the stoichiometric ratios of these components may be as described hereinbefore in respect of the third aspect of the invention. Further, reaction conditions employed in the second aspect of the invention may, where relevant, be the same as those employed in the third aspect of the invention (e.g. with respect to reaction time and temperature).

Step (II), if used, and step (III) of the second aspect of the invention preferably takes place when reaction between the compounds of formulae III and IV is substantially complete.

Adjustment of pH (i.e. step (II) of the second aspect of the invention) may be performed as hereinbefore described with respect to the first aspect of the invention (i.e. by the addition of a water-soluble acid, as hereinbefore defined, to the aqueous mixture obtained from step (I) above).

Also, formation of solid salt of formula I may be further promoted by cooling the mixture obtained from steps (I) and (II) and/or by adding a water-miscible alcohol, as defined hereinbefore.

For the avoidance of doubt, the term “monophasic aqueous solvent system”, when used herein, refers to a monophase with respect to solvents only. That is, this term is applied regardless of the physical forms of the components indicated hereinbefore as being reagents or products (even in the instances where these components are solids or oils that form separate phases from the aqueous solvent system).

A technical feature that is common to all of the first three aspects of the invention is the use of an aqueous solvent system to separate, in the presence of certain sulfonate anions, a protonated, “mono-substituted” oxabispidine from a protonated, “N,N-disubstituted” oxabispidine.

In this respect, and according to a fourth aspect of the invention, there is provided the use of an aqueous solvent system, as hereinbefore defined, in a method of isolating salt of formula I, as hereinbefore defined, which contains a cation of formula IIa

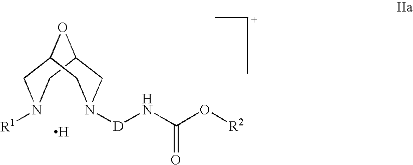

wherein D, R¹ and R² as hereinbefore defined, from a mixture comprising that cation and a cation of formula IIIa,

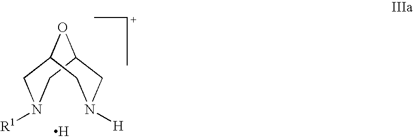

wherein R¹ is as hereinbefore defined, which method comprises:
(a) contacting the mixture of cations of formula IIa and iHIa with an aqueous solvent system, as hereinbefore defined, and a source of R³SO₃⁻ anions, wherein R³ is as hereinbefore defined;
(b) if necessary, adjusting the pH of the resulting mixture to any value from 3 to 8; and
(c) isolating the solid salt of formula I, or solvate thereof, thereby formed, which salt contains the cation of formula IIa.

As mentioned above, solvates of the compounds of formula I that may be mentioned include hydrates (e.g. monohydrates).

In this aspect of the invention, preferences for the salt of formula I, pH adjustment and sources of R³SO₃⁻ anions are the same as those set out above with respect to the first aspect of the invention. Further, it is preferred that the aqueous solvent system provides the only solvent(s) present in the mixture described at steps (a) and (b) above.

Those skilled in the art will appreciate that each of the cations of formulae Iha and IIIa will always be associated with a counter-anion, but that the cations and counter-anions may dissociate from one another when one and/or the other is solvated (e.g. in aqueous solution).

In this respect, a mixture of cations of formulae Ia and IIIa may be found, for example, in a mixture comprising two salts, one salt containing the cation of formula IIa and the other containing the cation of formula IIIa, with each cation being associated with one or more counter-anions. This mixture of salts may be utilised in the method according to the fourth aspect of the invention in the form of a mixture of solids or as a solution in an aqueous solvent system, as hereinbefore defined.

The method according to the fourth aspect of the invention envisages the mixture of salts comprising the cations of formulae Ia and IIIa as incorporating any one or more counter-anions, including, for example, halide, citrate and/or R³SO₃⁻ anions, wherein R³ is as hereinbefore defined. In a particularly preferred embodiment of the fourth aspect of the invention, however, the only anions present in the mixture described at (a) and (b) above are R³SO₃⁻ anions and, optionally, one or both of halide and citrate anions.

Compounds of formulae III and IV may be prepared in accordance with techniques known to those skilled in the art, such as those described in international patent applications WO 01/028992, WO 02/028864, WO 02/083690 and WO 2004/035592, the disclosures of which are hereby incorporated by reference.

For example, compounds of formula III may be prepared by dehydrative cyclisation of a compound of formula V,

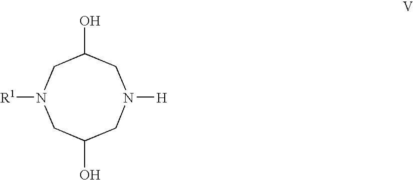

or a protected (e.g. N-benzenesulfonyl or N-nitrobenzenesulfonyl (e.g. N-4-nitrobenzenesulfonyl)) derivative thereof, wherein R¹ is as hereinbefore defined. The cyclisation may be carried out under conditions such as those described in WO 02/083690 (e.g. in the presence of a dehydrating agent, such as a strong acid (e.g. methanesulfonic acid or sulfuric acid), and a reaction-inert organic solvent (e.g. toluene or chlorobenzene)).

Compounds of formula III in which R¹ represents H or an amino protective group may alternatively be prepared according to, or by analogy with, known techniques, such as reaction of a compound of formula VI,

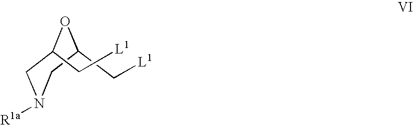

wherein R^{1a} represents H or an amino protective group (as hereinbefore defined) and L¹ represents a suitable leaving group (e.g. halo, such as iodo), with ammonia or a protected derivative thereof (e.g. benzylamine), for example under conditions such as those described in Chem. Ber. 96(11), 2827 (1963).

Compounds of formula III in which R¹ represents a structural fragment of formula Ia may alternatively be prepared by reaction of the compound of formula III in which R¹ represents H (i.e. the compound 9-oxa-3,7-diazabicyclo[3.3.1]nonane), or a derivative that is protected at the other nitrogen atom, with a compound of formula VII,

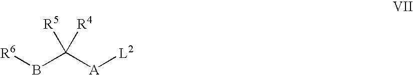

wherein L² represents a leaving group (e.g. mesylate, tosylate, mesitylenesulfonate or halo) and R⁴, R⁵, R⁶, A and B are as hereinbefore defined, for example under reaction conditions such as those described in WO 02/083690 (for example, at elevated temperature (e.g. between 35° C. and reflux temperature) in the presence of a suitable base (e.g. triethylamine or potassium carbonate) and an appropriate solvent (e.g. ethanol, toluene or water (or mixtures thereof)).

Compounds of formula III in which R¹ represents a structural fragment of formula Ia in which A represents C₂ alkylene and R⁴ and R⁵ together represent ═O may alternatively be prepared by reaction of 9-oxa-3,7-diazabicyclo[3.3.1]nonane, or a N-protected derivative thereof, with a compound of formula VIII,

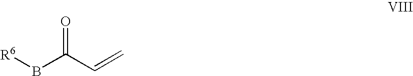

wherein R⁶ and B are as hereinbefore defined under, for example under reaction conditions such as those described in WO 02/083690 (for example, at room temperature in the presence of a suitable organic solvent (e.g. ethanol)).

Compounds of formula III in which R¹ represents a structural fragment of formula Ia in which A represents CH₂ and R⁴ represents —OH or —N(H)R⁸ may alternatively be prepared by reaction of 9-oxa-3,7-diazabicyclo[3.3.1]nonane, or a N-protected derivative thereof, with a compound of formula IX,

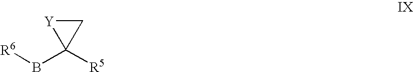

wherein Y represents —O— or —N(R⁸)— and R⁵, R⁶, R⁸ and B are as hereinbefore defined, for example under reaction conditions such as those described in WO 02/083690 (for example, at elevated temperature (e.g. between 60° C. and reflux) in the presence of a suitable solvent (e.g. water, isopropanol, ethanol or toluene (or mixtures thereof))).

Other compounds of formula III in which R¹ represents a structural fragment of formula Ia may alternatively be prepared by known techniques, for example according to techniques described in WO 01/028992, or by analogy with relevant processes known in the art for the introduction, and/or chemical conversion, of corresponding side-chains into, and/or in (as appropriate), corresponding bispidine compounds, for example as described in international patent application numbers WO 99/031100, WO 00/076997, WO 00/076998, WO 00/076999 and WO 00/077000, the disclosures in all of which documents are hereby incorporated by reference.

Compounds of formula IV may be prepared by reaction of a corresponding compound of formula X,

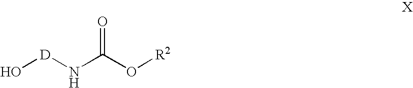

wherein D and R² are as hereinbefore defined, with a compound of formula XI,

R³—S(O)₂-L³ XI

wherein L³ represents a leaving group (e.g. halo, such as chloro) and R³ is as hereinbefore defined, for example under reaction conditions such as those described in WO 02/083690.

Compounds of formulae V, VI, VII, VIII, IX, X and XI, and derivatives thereof, are either commercially available, are known in the literature (e.g. the preparation of compounds of formulae V, VI, VII and IX is described in WO 02/083690) or may be obtained by conventional synthetic procedures, in accordance with known techniques, from readily available starting materials using appropriate reagents and reaction conditions.

As stated above, the process of the invention is preferably carried out to produce sulfonic acid salts of formula I in which R¹ represents an amino protective group, such as benzyl.

Salts of formula I in which R¹ represents an amino protective group may be further elaborated by neutralisation of the salt (i.e. liberation of the free base of formula II), removal of the amino protective group and then introduction of an R¹ group of formula Ia.

Thus, there is provided the following three further aspects of the invention.
(I) A process for the preparation of a compound of formula II, as hereinbefore defined, which process comprises a process as described hereinbefore for the preparation of a corresponding sulfonic acid salt of formula I, followed by neutralisation of that salt.
(II) A process for the preparation of a compound of formula II, as hereinbefore defined, in which R¹ represents H, which process comprises a process as described hereinbefore for the preparation of a corresponding sulfonic acid salt of formula I in which R¹ represents an amino protective group, followed by neutralisation of that salt and then removal of the amino protective group.
(III) A process for the preparation of a compound of formula II, as hereinbefore defined, in which R¹ represents:
   a) a structural fragment of formula Ia;
   b) a structural fragment of formula Ia, in which A represents C₂ alkylene and R⁴ and R⁵ together represent ═O; or
   c) a structural fragment of formula Ia, in which A represents CH₂ and R⁴ represents —OH or —N(H)R⁸,
   which process comprises a process according to either of (I) and (II) above for the preparation of a corresponding compound of formula II in which R¹ represents H, followed by reaction of that compound with, respectively
   1) a compound of formula VII, as hereinbefore defined,
   2) a compound of formula VIII, as hereinbefore defined, or
   3) a compound of formula IX, as hereinbefore defined.

In process (III) above, preferred values of R¹ (the structural fragment of formula Ia), include the preferred values of the fragment of formula Ia detailed above with respect to the sulfonic acid salt of formula I.

In these further aspects of the invention, neutralisation and removal of amino protective groups may be carried out under conditions known to the skilled person, such as those described in WO 02/083690. For example, neutralisation may be effected by reaction with a base (e.g. an alkali metal hydroxide, carbonate or hydrogencarbonate). Further, when the amino protective group is benzyl, then that group may be removed by hydrogenation in the presence of an appropriate catalyst (e.g. Pd/C or Pt/C).

Also, coupling between a compound of formula II in which R¹ represents H with a compound of formula VII, VIII or IX may be performed under conditions described hereinbefore with respect to the preparation of compounds of formula II.

In addition to these further aspects of the invention described above, the skilled person will appreciate that certain compounds of formula I or II may be prepared from certain other compounds of formula I or II, respectively, or from structurally related compounds. For example, compounds of formula I or II in which R¹ represents certain structural fragments of formula Ia may be prepared, in accordance with relevant processes known in the art, by the respective interconversion of corresponding compounds of formula I or II in which R¹ represents an amino protective group or different structural fragments of formula Ia (for example by analogy with the processes described in international patent application numbers WO 99/031100, WO 00/076997, WO 00/076998, WO 00/076999, WO 00/077000 and WO 01/028992).

It will be appreciated by those skilled in the art that, in the processes described above, the functional groups of intermediate compounds may be, or may need to be, protected by protecting groups.

In any event, functional groups which it is desirable to protect include hydroxy and amino. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include the amino protective groups mentioned hereinbefore, such as benzyl, sulfonyl (e.g. benzenesulfonyl or 4-nitrobenzenesulfonyl), tert-butyloxycarbonyl, 9-fluorenyl-methoxycarbonyl or benzyloxycarbonyl.

The protection and deprotection of functional groups may take place before or after any of the reaction steps described hereinbefore.

Protecting groups may be removed in accordance with techniques which are well known to those skilled in the art and as described hereinafter.

The use of protecting groups is described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

The process of the invention may have the advantage that the salt of formula I, or solvate thereof, is selectively isolated in high purity from a mixture containing a number of unwanted organic and inorganic materials.

In particular, the process of the invention may also have the advantage that the salt of formula I may, directly from the reaction mixture in which it is formed, be obtained via a controlled crystallisation step. This allows the salt of formula I to be prepared in high yield, acceptable purity and/or in a form that is easy to handle by way of a process that avoids the further purification procedures that would be rendered necessary by the processes of the prior art (i.e. by way of a process that involves a reduced number of unit operations compared to the processes of the prior art).

Further, the process of the invention may also have the advantage that the salt of formula I is produced in higher yield, in higher purity, in less time, in a more convenient (i.e. easy to handle) form, from more convenient (i.e. easy to handle) precursors, at a lower cost and/or with less usage and/or wastage of materials (including reagents and solvents) compared to the procedures disclosed in the prior art.

“Substantially”, when used herein, may mean greater than 50%, preferably greater than 75%, for example greater then 95%, and particularly greater than 99%.

The term “relative volume” (rel. vol.), when used herein, refers to the volume (in millilitres) per gram of reagent employed.

The invention is illustrated, but in no way limited, by the following examples.

All relative volumes (rel. vol.) and equivalents (eq.) in the following example are measured with respect to the amount of 3-benzyl-9-oxa-3,7-diazabicyclo[3.3.1]nonane dihydrochloride used.

### EXAMPLE 1

### [2-(7-Benzyl-9-oxa-3,7-diazabicyclo[3.3.1]non-3-yl)ethyl]carbamic acid tert-butyl ester, 2,4,6-trimethylbenzenesulfonic acid salt monohydrate

### Alternative I

Solid 3-benzyl-9-oxa-3,7-diazabicyclo[3.3.1]nonane dihydrochloride (200.2 g, 1.0 eq.; see WO 02/083690), aqueous sodium hydroxide (2.5 M, 900 mL, 4.5 rel. vol.) and solid 2-(tert-butyloxycarbonylamino)ethyl 2,4,6-trimethylbenzene-sulfonate (248.4 g, 1.05 eq.; see WO 02/083690) were charged to a reaction vessel. Stirring was started, toluene (500 mL, 2.5 rel. vol.) was charged and the reaction heated from 27° C. to 65° C. over 20 minutes. The reaction was held at 65° C.±5° C. for 12 hours and then stirred at ambient temperature for 8 hours and left to stand for 24 hours. The mixture was reheated to 65° C. and the stirring stopped. The lower aqueous layer (first aqueous phase) was separated and added to a mixture of water (900 mL, 4.5 rel. vol.) and isopropanol (400 mL, 2 rel. vol.) thereby producing diluted first aqueous phase.

The temperature of the upper toluene layer (first organic phase) that was left in the original reaction vessel was noted to be 60° C. A cold (20° C.) solution of aqueous citric acid (10% w/v, 1000 mL, 5 rel. vol.) was then added to this toluene phase. The resulting mixture had a temperature of 38° C. This mixture was stirred for 5 minutes and then the stirring stopped to give an upper organic phase and a lower aqueous phase (second aqueous phase). These phases were separated and the organic phase only was discarded. The diluted first aqueous phase was heated to 75° C. The second aqueous phase was then added at such a rate that the temperature remained above 70° C. (this took 22 minutes). The mixture was stirred at 75° C. for 1 hour, then allowed to cool to 41° C. over 4 hours. The mixture was then stirred for 65 hours. The mixture, now at 23° C., was filtered. The filter cake was washed by displacement with water (800 mL, 4 rel. vol., water temperature was 22° C.) and then cold isopropanol (800 mL, 4 rel. vol., IPA temperature was 5° C.). The cake was sucked dry on the filter for 40 minutes, then the solid transferred to a vacuum oven. The solid was dried to constant weight in vacuo at 50° C. for 20 hours. This gave the title compound as a white solid (346.3 g, 90%).

Water by KF analysis=3.4% (monohydrate requires 3.1%)

¹H-NMR (400 MHz, CDCl₃) δ 1.44 (9H, s), 2.23 (3H, s), 2.73 (6H, s), 2.74-2.90 (5H, m), 2.95-3.0 (4H, m), 3.4-3.45 (2H, m), 3.65-3.70 (4H, m), 4.19 (2H, s), 4.30 (2H, s), 6.84 (2H, s), 6.95 (1H, bs), 7.40 (5H, s).

¹H-NMR (400 MHz, DMSO-d₆) δ 1.43 (9H, s), 2.17 (3H, s), 2.75 (2H, t), 2.90-2.94 (4H, m), 3.14-3.22 (4H, m), 3.22-3.4 (6H, m), 3.89 (2H, s), 4.13 (2H, s), 6.74 (2H, s), 7.12 (1H, bs), 7.42-7.46 (5H, m).

### Alternative II

Solid 3-benzyl-9-oxa-3,7-diazabicyclo[3.3.1]nonane dihydrochloride (100.1 g, 1.0 eq.; see WO 02/083690), was added to aqueous sodium hydroxide (44 g of solid NaOH dissolved in 394 g of water) that was in a reaction vessel. At 25° C., solid 2-(tert-butyloxycarbonylamino)ethyl 2,4,6-trimethylbenzenesulfonate (124.0 g, 1.05 eq.; see WO 02/083690) was charged to the reaction vessel. Stirring was started, toluene (100 g, 1.0 rel wt.) was charged and the reaction heated from 25° C. to 65° C.±3° C. over 10 minutes. The reaction was held at 65° C.±3° C. for 7 hours. Stirring was stopped and the lower aqueous layer (first aqueous phase) was separated at 60-65° C. (a small amount of interfacial material was kept with the organic phase), and added to a mixture of water (450 g, 4.5 rel wt) and isopropanol (150 g, 1.5 rel wt.), thereby producing a diluted, first aqueous phase. The temperature of the upper toluene layer that was left in the original reaction vessel (first organic phase) was noted to be 60° C. A cold (20° C.) solution of aqueous citric acid (10% w/w, 500 g, 5 rel wt.) was added to the toluene phase. The resulting mixture had a temperature of 40° C. This mixture was stirred for 5 minutes and then the stirring stopped to give an upper organic phase and a lower aqueous phase (second aqueous phase). These phases were separated and the organic phase only was discarded.

The diluted, first aqueous phase was heated to 75° C. The second aqueous phase was then added to the warmed, diluted, first aqueous phase such that the temperature was maintained in the range of 75° C.±5° C. (this took 54 minutes). The mixture was stirred at 75° C.±5° C. for 1 hour 18 minutes, before being allowed to cool naturally from 72° C. to 68° C. over 13 minutes (a lot of precipitate formed in this time). The slurry was then allowed to cool naturally from 68° C. to 40° C. over 2 hours, after which it was cooled in an ice/water bath from 40° C. to 5° C. over 47 minutes and then stirred at 5° C. for 1 hour. The mixture was filtered and the filter cake washed by displacement with cold (5° C.) water (400 g, 4.0 rel vol), then cold (5° C.) isopropanol (300 g, 3.0 rel wt). The filter cake was dried by suction on the filter for 37 minutes, before being transferred to a dish and left to air dry overnight. The resulting solid (195 g) was then dried to constant weight in vacuo at 50° C. for 6 hours 30 minutes. This gave the title compound as a white solid (176.50 g, 91%).

Water by KF analysis=3.26% (monohydrate requires 3.1%)

### Alternative III

Solid 3-benzyl-9-oxa-3,7-diazabicyclo[3.3.1]nonane dihydrochloride (100 g, 1.0 eq.; see WO 02/083690), aqueous sodium hydroxide (2.5 M, 450 mL, 4.5 rel vol) and solid 2-(tert-butyloxycarbonylamino)ethyl 2,4,6-trimethylbenzene-sulfonate (117.86 g, 1.0 eq.; see WO 02/083690) were charged to a reaction vessel. Stirring was started and the reaction heated to 65° C.±5° C. for 6 hours. At this point, isopropanol (200 mL, 2 rel. vol.) and water (400 mL, 4 rel. vol.) were added to the reaction mixture, which was then heated to 75° C. Citric acid (10% w/v, 500 mL, 5 rel. vol.) was added slowly, such that the temperature was maintained above 70° C. During the addition of citric acid, product was noted to precipitate from solution. The resulting mixture was allowed to cool slowly to room temperature, at which temperature it was stirred overnight. The solid product was isolated by filtration, and washed with water (3×200 mL, 6 rel. vol.) on the filter. The filter cake was then washed with cold isopropanol (200 mL, 2 rel. vol.), before being dried by suction on the filter and then transferred to a vacuum oven. The product was dried to constant weight in vacuo at 50° C. for 20 hours. This gave the title compound as a white solid (168 g, 87%).

Water by KF analysis=3.17% (monohydrate requires 3.1%)

### Alternative IV

A solution of 20% w/w aqueous sodium hydroxide (1.10 moles; 220.00 g), which was at 22° C., was added to a 2 L flask with stirring at 300 rpm. Water (24.98 moles; 450.00 mL; 450.00 g), which was at 22° C., was then added. The final temperature of the resulting mixture was 23° C. Solid 3-benzyl-9-oxa-3,7-diazabicyclo[3.3.1]nonane dihydrochloride (1.00 eq.; 343.38 mmoles; 100.00 g; see WO 02/083690) was added, at which point the temperature of the mixture rose to 26° C. Solid 2-(tert-butyloxycarbonylamino)ethyl 2,4,6-trimethylbenzene-sulfonate (1.05 eq.; 361.05 mmoles; 124.00 g; see WO 02/083690) was added (no temperature change due to this addition was observed). Toluene (2.17 moles; 231.21 mL; 200.00 g), which was at 22° C., was then added, which caused the temperature of the mixture to fall to 23° C. The mixture was heated from 23° C. to 65° C.±5° C. in 16 minutes and then held at this temperature for 6 hours 20 minutes. Stirring was stopped and the phases were allowed to settle (this took 55 seconds). The aqueous phase (first aqueous phase) was separated from the organic phase, keeping interfacial material with the organic phase. The temperature of the phases at separation was ca. 54° C. Under stirring, a solution of 10% w/w aqueous citric acid (260.25 mmoles; 500.00 g) was added to the toluene phase, to provide a mixture having a temperature of 40° C. The temperature of the mixture was then adjusted to 45° C., at which temperature stirring was stopped and the phases allowed to settle (this took 49 seconds). The resulting aqueous phase (second aqueous phase) was separated from the organic phase, leaving interfacial material with the organic phase. The organic phase was then discarded. Isopropanol (2.50 moles; 191.08 mL; 150.00 g), which was at 22° C., was added to the first aqueous phase (which was then at 49° C.) to provide a mixture having a temperature of 47° C. The second aqueous phase, which was then at 43° C., was added to the diluted first aqueous phase (at this point having a temperature of 44° C.) over the course of 50 seconds. This provided a mixture having a final temperature of 47° C. During the addition, a precipitate formed that ultimately hindered stirring in the vessel. The stirring rate was increased to 400 rpm and the mixture was heated to 72° C.±3° C. At 62° C., the mixture became stirrable. Upon reaching 72° C., the stirring rate was reduced to 350 rpm and the mixture was held at 72° C.±3° C. for 30 minutes before being allowed to cool overnight. The mixture was then cooled from 22° C. to 5° C. over the course of 1 hour, before being held at 5° C. for 55 minutes. The product was collected by filtration (15 cm diameter Büchner fimel), which took 65 seconds. The product cake was washed with cold (5° C.) water (22.20 moles; 400.00 mL; 400.00 g), which took 35 seconds. The product cake was next washed with cold (5° C.) isopropanol (4.99 moles; 382.17 mL; 300.00 g), which took 60 seconds (if desired, this isopropanol wash can be omitted to increase yield but potentially decrease product purity). The cake was sucked as dry as possible over 90 minutes, after which the resulting, damp solid (236 g) was dried in vacuo (at 70° C. for 5 hours) to give the title compound as a white solid (174.4 g, 90.4%). If desired, a longer drying period (e.g. 59 hours) at 70° C. in vacuo can be utilised to provide a solid with lower water content (water content approximately 0.3% w/w).

Water by KF analysis=2.8% w/w (monohydrate requires 3.1% w/w).

Alternative cooling profiles can be applied to the mixture (of first and second aqueous phases) in order to improve stirring properties of the mixture as well as filtration and washing properties, for example, as follows.

After cooling the reaction mixture (for convenience) to room temperature overnight, the mixture was heated to 80° C., with stirring at 500 rpm. The mixture was then:

(i) cooled, over the course of 60 minutes, to 70° C.;
(ii) heated from 70° C. to 75° C. over the course of 30 minutes;
(iii) cooled from 75° C. to 65° C. over the course of 60 minutes; and
(iv) cooled from 65° C. to 5° C. over the course of 120 minutes.

The resulting mixture was then held at 5° C. for 2 hours. The product was collected by filtration then washed and dried as above.

### EXAMPLE 2

### [2-(7-Benzyl-9-oxa-3,7-diazabicyclo[3.3.1]non-3-yl)ethyl]carbamic acid tert-butyl ester 2,4,6-trimethylbenzenesulfonic acid salt anhydrate

Solid 3-benzyl-9-oxa-3,7-diazabicyclo[3.3.1]nonane dihydrochloride (55.0 kg, 1.0 eq.; see WO 02/083690) and aqueous sodium hydroxide (2.5 M, 270.3 kg, 4.5 rel. vol.) were charged to a reaction vessel (VESSEL 1). Toluene (79.0 kg, 1.66 rel. vol.) was added and stirring was started. Solid 2-(tert-butyloxycarbonyl-amino)ethyl 2,4,6-trimethylbenzenesulfonate (71.5 kg, 1.10 mol. eq.; see WO 02/083690) was charged to a second vessel (VESSEL 2) and toluene (171.0 kg, 3.59 rel. vol.) added. Stirring was started and the mixture heated to 29.3° C. over 44 minutes to form a solution. The solution at 29.3° C. in VESSEL 2 was then added to the mixture in VESSEL 1. VESSEL 2 was then charged with toluene (45 kg, 0.95 rel. vol.), heated to 29.7° C. and then added to the mixture in VESSEL 1. The mixture in VESSEL 1 was heated to 66.0° C. over 28 minutes with stirring and held at this temperature for 17 hours 55 minutes. Stirring was stopped and the phases allowed to separate over 66 minutes and the lower aqueous phase (first aqueous phase) sent to a vessel (VESSEL 3) at 64.4° C. Demineralised water (137.5 kg, 2.5 rel. vol.) and isopropanol (86.7 kg, 2 rel. vol.) were added to VESSEL 3, giving diluted first aqueous phase, the temperature of which was adjusted to 35° C. The organic phase (first organic phase) retained in VESSEL 1 was cooled to 17.4° C. and aqueous citric acid solution (0.5 M, 275.0 kg, 5 rel. vol.) was added and stirred for 36 minutes. The stirring was stopped and the phases allowed to separate for 25 minutes. The lower aqueous phase (second aqueous phase) was separated to a vessel (VESSEL 4) and the upper organic phase was discarded. The first aqueous phase (in VESSEL 3) was heated to 75.6° C. and the second aqueous phase added to it over 47 minutes (at such a rate so as to maintain the temperature in VESSEL 3 above 70° C. VESSEL 4 was charged with demineralised water (109.7 kg, 2 rel. vol.) and rinsed into the mixture in VESSEL 3. The mixture (initially observed to be at 73.3° C.) was then cooled to 20.6° C. over 4 hours 17 minutes, before being stirred for 10 hours 33 minutes (this time was used for convenience, as 4 hours is sufficient). The mixture was then filtered to give a solid. A displacement wash with demineralised water (330.4 kg, 6 rel. vol.) was carried out. The solid was then dried on the filter by applying vacuum and then by heating at 50° C. for 66 hours. This gave the title compound as a damp white solid (104.40 kg discharged, dry weight equivalent 92.26 kg, 87%).

### EXAMPLE 3

The materials produced by Examples 1 and 2 above were analysed by HPLC for content of 3-benzyl-9-oxa-3,7-diazabicyclo[3.3.1]nonane (i.e. unreacted starting material), and were found to contain less that 0.075% (by HPLC peak area, as measured at 220 nm) of that material.

### ABBREVIATIONS

bs=broad (in relation to NMR)
DMSO=dimethylsulfoxide
Et=ethyl
eq.=equivalents
IPA=iso-propyl alcohol (isopropanol)
m=multiplet (in relation to NMR)
Me=methyl
min.=minute(s)
mol.=molar
Pd/C=palladium on carbon
Pt/C=platinum on carbon
s=singlet (in relation to NMR)
t=triplet (in relation to NMR)

Prefixes n-, s-, i-, t- and tert- have their usual meanings: normal, secondary, iso, and tertiary.

1. A process for isolating a salt of formula I, 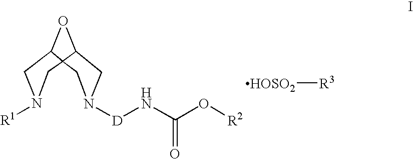
or a solvate thereof,
wherein
R¹ represents H, an amino protective group or a structural fragment of formula Ia, 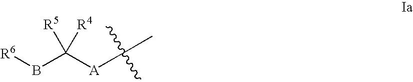
in which
R⁴ represents H, halo, C₁₋₆ alkyl, —OR⁷, -E-N(R⁸)R⁹ or, together with R⁵, represents ═O;
R⁵ represents H, C₁₋₆ alkyl or, together with R⁴, represents ═O;
R⁷ represents H, C₁₋₆ alkyl, -E-aryl, -E-Het¹, —C(O)R^{10a}, —C(O)OR^{10b} or —C(O)N(R^{11a})R^{11b};
R⁸ represents H, C₁₋₆ alkyl, -E-aryl, -E-Het¹, —C(O)R^{10a}, —C(O)OR^{10b}, —S(O)₂R^{10c}, —[C(O)]ₚN(R^{11a})R^{11b} or —C(NH)NH₂;
R⁹ represents H, C₁₋₆ alkyl, -E-aryl or —C(O)R^{10d};
R^{10a} to R^{10d} independently represent, at each occurrence when used herein, C₁₋₆ alkyl (optionally substituted by one or more substituents selected from halo, aryl and Het²), aryl, Het³, or R^{10a} and R^{10d} independently represent H;
R^{11a} and R^{11b} independently represent, at each occurrence when used herein, H or C₁₋₆ alkyl (optionally substituted by one or more substituents selected from halo, aryl and Het⁴), aryl, Het⁵, or together represent C₃₋₆ alkylene, optionally interrupted by an O atom;
E represents, at each occurrence when used herein, a direct bond or C₁₋₄ alkylene;
p represents 1 or 2;
A represents a direct bond, -J-, -J-N(R^{12a})—, -J-S(O)₂N(R^{12b})—, -J-N(R^{12c})S(O)₂— or -J-O— (in which latter four groups, -J is attached to the oxabispidine ring nitrogen);
B represents -Z-{[C(O)]ₐC(H)(R^{13a})}_{b}-, -Z-[C(O)]_{c}N(R^{13b})—, -ZN(R^{13c})S(O)₂—, -Z-S(O)₂N(R^{13d})—, -Z-S(O)ₙ—, -Z-O— (in which latter six groups, Z is attached to the carbon atom bearing R⁴ and R⁵), N(R^{13e})-Z-, —N(R^{13f})S(O)₂-Z-, —S(O)₂N(R^{13g})-Z or —N(R^{13h})C(O)O_Z (in which latter four groups, Z is attached to the R⁶ group);
J represents C₁₋₆ alkylene optionally interrupted by —S(O)₂N(R^{12d})— or —N(R^{12e})S(O)₂— and/or optionally substituted by one or more substituents selected from —OH, halo and amino;
Z represents a direct bond or C₁₋₄ alkylene, optionally interrupted by —N(R¹³ⁱ)S(O)₂— or —S(O)₂N(R^{13j})—;
a, b and c independently represent 0 or 1;
n represents 0, 1 or 2;
R^{12a} to R^{12e} independently represent, at each occurrence when used herein, H or C₁₋₆ alkyl;
R^{13a} represents H or, together with a single ortho-substituent on the R⁶ group (ortho-relative to the position at which the B group is attached), R^{13a} represents C₂₋₄ alkylene optionally interrupted or terminated by O, S, N(H) or N(C₁₋₆ alkyl);
R^{13b} represents H, C₁₋₆ alkyl or, together with a single ortho-substituent on the R⁶ group (ortho-relative to the position at which the B group is attached), R^{13b} represents C₂₋₄ alkylene;
R^{13c} to R¹³ⁱ independently represent, at each occurrence when used herein, H or C₁₋₆ alkyl;
R⁶ represents phenyl or pyridyl, both of which groups are optionally substituted by one or more substituents selected from —OH, cyano, halo, nitro, C₁₋₆ alkyl (optionally terminated by —N(H)C(O)OR^{14a}), C₁₋₆ alkoxy, —N(R^{15a})R^{15b}, —C(O)R^{15c}, —C(O)OR^{15d}, —C(O)N(R^{15e})R^{15f}, —N(R^{15g})C(O)R^{15h}, —N(R¹⁵ⁱ)C(O)N(R^{15j})R^{15k}, —N(R^{15m})S(O)₂R^{14b}, —S(O)₂N(R¹⁵ⁿ)R^{15o}, —S(O)₂R^{14c}, —OS(O)₂R^{14d} and/or aryl;
and an ortho-substituent (ortho-relative to the attachment of B) may
(i) together with R^{13a}, represent C₂₋₄ alkylene optionally interrupted or terminated by O, S, N(H) or N(C₁₋₆ alkyl), or
(ii) together with R^{13b}, represent C₂₋₄ alkylene;
R^{14a} to R^{14d} independently represent C₁₋₆ alkyl;
R^{15a} and R^{15b} independently represent H, C₁₋₆ alkyl or together represent C₃₋₆ alkylene, resulting in a four- to seven-membered nitrogen-containing ring;
R^{15c} to R^{15o} independently represent H or C₁₋₆ alkyl; and
Het¹ to Het⁵ independently represent, at each occurrence when used herein, five- to twelve-membered heterocyclic groups containing one or more heteroatoms selected from oxygen, nitrogen and/or sulfur, which heterocyclic groups are optionally substituted by one or more substituents selected from ═O, —OH, cyano, halo, nitro, C₁₋₆ alkyl, C₁₋₆ alkoxy, aryl, aryloxy, —N(R^{16a})R^{16b}, —C(O)R^{16c}, —C(O)OR^{16d}, —C(O)N(R^{16e})R^{16f}, N(R^{16g})C(O)R^{16h}, —S(O)₂N(R¹⁶ⁱ)(R^{16j}) and/or —N(R^{16k})S(O)₂R^{16l};
R^{16a} to R^{16l} independently represent C₁₋₆ alkyl, aryl or R^{16a} to R^{16k} independently represent H;
provided that:
(a) when R⁵ represents H or C₁₋₆ alkyl; and A represents -J-N(R^{12a})— or -J-O—, then:
(i) J does not represent C₁ alkylene or 1,1-C₂₋₆ alkylene; and
(ii) B does not represent —N(R^{13b})—, —N(R^{13c})S(O)₂—, —S(O)ₙ—, —O—, —N(R^{13e})-Z, —N(R^{13f})S(O)₂-Z- or —N(R^{13h})C(O)O-Z-;
(b) when R⁴ represents —OR⁷ or -E-N(R³)R⁹ in which E represents a direct bond, then:
(i) A does not represent a direct bond, -J-N(R^{12a})—, -J-S(O)₂N(R^{12b})— or -J-O—; and
(ii) B does not represent —N(R^{13b})—, —N(R^{13c})S(O)₂—, —S(O)ₙ—, —O—, —N(R^{13e})-Z, —N(R^{13f})S(O)₂-Z- or —N(R^{13h})C(O)O-Z-;
(c) when A represents -J-N(R^{12c})S(O)₂—, then J does not represent C₁ alkylene or 1,1-C₂₋₆ alkylene; and
(d) when R⁵ represents H or C₁₋₆ alkyl and A represents -J-S(O)₂N(R^{12b})—, then B does not represent —N(R^{13b})—, —N(R^{13c})S(O)₂—, —S(O)ₙ—, —O—, —N(R^{13e})-Z-, —N(R^{13f})S(O)₂-Z- or —N(R^{13h})C(O)O-Z-; and
D represents optionally branched C₂₋₆ alkylene, provided that D does not represent 1,1-C₂₋₆ alkylene;
R² represents C₁₋₆ alkyl (optionally substituted by one or more substituents selected from —OH, halo, cyano, nitro and aryl) or aryl; and
R³ represents unsubstituted C₁₋₄ alkyl, C₁₋₄ perfluoroalkyl or phenyl, which latter group is optionally substituted by one or more substituents selected from C₁₋₆ alkyl, halo, nitro and C₁₋₆ alkoxy;
wherein each aryl and aryloxy group, unless otherwise specified, is optionally substituted;
from a mixture comprising a compound of formula II, 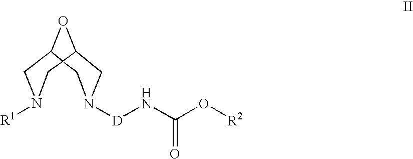
wherein D, R¹ and R² are as defined above, and a compound of formula ITT, 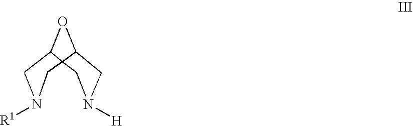
or a salt and/or a solvate thereof, wherein R¹ is as defined above;
which process comprises:
(1) providing, in an aqueous solvent system, a dispersion of
(i) the compounds of formulae II and III, as defined above and
(ii) a source of R³SO₃⁻ anions, wherein R³ is as defined above;
(2) if necessary, adjusting the pH of the aqueous dispersion to any value from 3 to 8; and
(3) isolating the solid salt of formula I, or solvate thereof, thereby formed.

2. A process as clamed in claim 1, wherein the salt is of formula Ib, 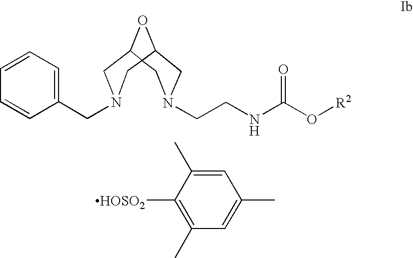
or a hydrate thereof
wherein R² is as defined in claim 1.

3. A process as claimed in claim 1, wherein R² represents tert-butyl.

4. A process as claimed in claim 1, wherein the mixture of compounds of formulae II and III is obtained by incomplete reaction of a compound of formula ITT, as defined in claim 1, or a salt and/or solvate thereof, with a compound of formula IV, 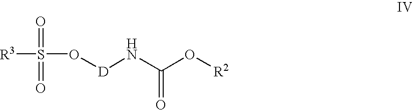
wherein D, R² and R³ are as defined in claim 1, in the presence of solvent and base.

5. A process as claimed in claim 4, wherein the mixture of compounds of formulae II and III is obtained by incomplete reaction of the compounds of formulae III and IV in the presence of an aqueous phase and base.

6. A process as claimed in claim 4, wherein R³SO₃⁻ anions present in the dispersion of step (1) are derived from the compound of formula IV.

7. A process for preparing a salt of formula I, as defined in claim 1, or a solvate thereof, which process comprises:
(A) effecting reaction between base, a compound of formula ITT, as defined in claim 1, or a salt and/or solvate thereof and a compound of formula IV, 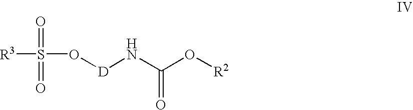
wherein D, R² and R³ are as defined in claim 1, in the presence of base and a solvent system that is part-aqueous and biphasic;
(B) separating the first organic and first aqueous phases that are obtained after performance of step (A), and retaining both of these phases;
(C) extracting the first organic phase with an aqueous solution of an acid to produce a second aqueous phase;
(D) separating the second aqueous phase and then combining it with the first aqueous phase to produce a precipitation mixture;
(E) if necessary, adjusting the pH of the precipitation mixture to any value from 3 to 8; and then
(F) isolating the solid salt of formula I, or solvate thereof, thereby formed.

8. A process as claimed in claim 7, wherein step (A) comprises effecting incomplete reaction between base, a compound of formula III, or a salt and/or solvate thereof and a compound of formula IV, in the presence of base and a solvent system that is part-aqueous and biphasic.

9. A process as claimed in claim 7, wherein the organic solvent of the biphasic solvent system is an aromatic hydrocarbon.

10. A process as claimed in claim 9, wherein the organic solvent is toluene.

11. A process as claimed in claim 7, wherein the compound of formula III is employed in acid addition salt form.

12. A process as claimed in claim 11, wherein the compound of formula III is employed in the form of a dihydrochloride salt.

13. A process as claimed in claim 7, wherein the base is an alkali metal hydroxide.

14. A process as claimed in claim 13, wherein the base is sodium hydroxide.

15. A process as claimed in claim 7, wherein the acid employed in step (C) is a weak, water-soluble acid.

16. A process as claimed in claim 15, wherein the acid is citric acid.

17. A process as claimed in claim 7, wherein, when the first and second aqueous phases are combined, additional water and/or a water-miscible alcohol is added so that it is present in the resulting precipitation mixture.

18. A process as claimed in claim 17, wherein the water-miscible alcohol is isopropanol.

19. A process as claimed in claim 1 comprising the further step of recrystallising the salt of formula I from a mixture of water and isopropanol.

20. A process for the preparation of a compound of formula II, as defined in claim 1, which process comprises a process as defined in claim 1 for the preparation of a corresponding sulfonic acid salt of formula I, followed by neutralisation of that salt.

21. A process for the preparation of a compound of formula II, as defined in claim 1, wherein R¹ represents H, which process comprises a process as defined in claim 1 for the preparation of a corresponding sulfonic acid salt of formula I in which R¹ represents an amino protective group, followed by neutralisation of that salt and then removal of the amino protective group.

22. A process for the preparation of a compound of formula II, as defined in claim 1, in which R¹ represents:
a) a structural fragment of formula Ia;
b) a structural fragment of formula Ia in which A represents C₂ alkylene and R⁴ and R⁵ together represent ═O; or
c) a structural fragment of formula Ia in which A represents CH₂ and R⁴ represents —OH or —N(H)R⁸,
which process comprises a process for the preparation of a corresponding compound of formula II in which R¹ represents H, wherein said process comprises a process as defined in claim 1 for the preparation of a corresponding sulfonic acid salt of formula I, followed by neutralisation of that salt or a process as defined in claim 1 for the preparation of a corresponding sulfonic acid salt of formula I in which R¹ represents an amino protective group followed by neutralisation of that salt and then removal of the amino protective uouP followed by reaction of that compound with, respectively
1) a compound of formula VII, 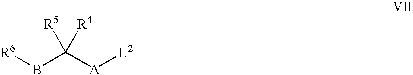
wherein L² represents a leaving group and R⁴, R⁵, R⁶, A and B are as defined in claim 1,
2) a compound of formula VIII, 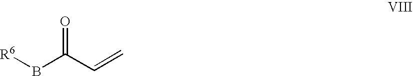
wherein R⁶ and B are as defined in claim 1, or
3) a compound of formula IX, 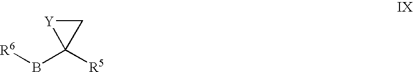
wherein Y represents —O— or —N(R⁸)— and R⁵, R⁶, R⁸ and B are as defined in claim 1.

23. A process as claimed in claim 22, wherein the structural fragment of formula Ia in the compound of formula II that is ultimately produced represents:

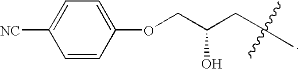

24. A process as claimed in claim 22, wherein the structural fragment of formula Ia in the compound of formula II that is ultimately produced represents:

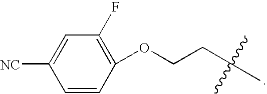

25. A process as claimed in claim 22, wherein the structural fragment of formula Ia in the compound of formula II that is ultimately produced represents:

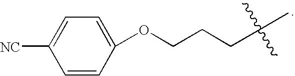

26. A process as claimed in claim 2, wherein R² represents tert-butyl.

27. A process as claimed in claim 5, wherein R³SO₃⁻ anions present in the dispersion of step (1) are derived from the compound of formula IV.

28. A process as claimed in claim 8, wherein the organic solvent of the biphasic solvent system is an aromatic hydrocarbon.
